(19) United States
(12) Patent Application Publication
(10) Pub. No.: US 2010/0280487 A1
(43) Pub. Date: Nov. 4, 2010

(54) METHOD OF REMOVING A STYLETTE FROM A CATHETER

(75) Inventors: Ramakrishna Venugopalan, Holliston, MA (US); Greg M. Schorn, Milford, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: CODMAN & SHURTLEFF, INC., RAYNHAM, MA (US)

(21) Appl. No.: 12/834,572

(22) Filed: Jul. 12, 2010

Related U.S. Application Data
(63) Continuation of application No. 11/262,697, filed on Oct. 31, 2005.

(51) Int. Cl. A61M 25/01; A61B 17/34

(52) U.S. Cl. 604/506; 604/164.01

(57) ABSTRACT

A hydrophilic coated stylette is pre-loaded in an implantable catheter. The hydrophilic coated stylette is wetted with saline to activate the hydrophilic coating. The stylette loaded catheter is tunneled, typically through muscle and tissue, to a desired target site within the body. The hydrophilic coated stylette is then removed from the catheter while maintaining the catheter intact. The proximal end of the catheter can be connected to an outlet of an implantable infusion pump so that medicament can be delivered directly to the target site.

### BACKGROUND OF THE INVENTION

1. Field of the Invention

The present invention relates to a method of removing a stylette from a catheter. More particularly, the present invention relates to a method of removing a stylette from a catheter with the use of a hydrophilic coated stylette.

2. Discussion of Related Art

It is well known in the art to place a catheter at a desired target site within the body. To aid in inserting the catheter, which often has to tunnel through muscle and tissue, a stylette or guide wire (hereinafter referred to as a stylette) may be used to help provide rigidity during the insertion process. Once the catheter is placed at a desired site, the stylette is removed. But in this process, it is sometimes difficult to remove the stylette. The stylette sticks to the catheter and can cause, among other things, displacement of the distal end of the catheter from the target site, and/or cutting of the catheter.

Some have attempted to solve this problem by coating the stylette with polytetrafluoroethylene (“PTFE”) to ease its removal. However, users continue to be dissatisfied with this attempted solution because the stylette still sometimes sticks to the catheter.

Published U.S. Patent Application No. 2005/0100580, entitled Hydrophilic Coated Medical Device teaches that a hydrophilic coating allows for easy insertion of wire guides during cardiac catherization. However, there is no teaching or suggestion of coating a guide wire with a hydrophilic material to aid in the removal of a stylette after a catheter has been tunneled into place at a desired site within a body.

Thus, there is a need in the art for a method of more effectively removing a stylette from a catheter, especially after the catheter has been placed at a desired site within a body.

### SUMMARY OF THE INVENTION

In accordance with a currently preferred exemplary embodiment, the present invention involves a hydrophilic coated stylette that is pre-loaded in an implantable catheter. The hydrophilic coated stylette is wetted with saline to activate the hydrophilic coating. The stylette loaded catheter is tunneled, typically through muscle and tissue, to a desired target site within the body. The hydrophilic coated stylette is then removed from the catheter, while maintaining the catheter intact. The proximal end of the catheter can be connected to an outlet of an implantable infusion pump so that medicament can be delivered directly to the target site.

### BRIEF DESCRIPTION OF THE DRAWINGS

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIG. 1 is a perspective view of a hydrophilic coated stylette loaded in a catheter with a syringe connected to a proximal end of the catheter;

FIG. 2 is a cross-sectional view taken along line 2-2 of FIG. 1 and looking in the direction of the arrows; and

FIG. 3 is a cross-sectional view taken along line 3-3 of FIG. 2 and looking in the direction of the arrows.

### DETAILED DESCRIPTION OF THE CURRENTLY PREFERRED EXEMPLARY EMBODIMENT

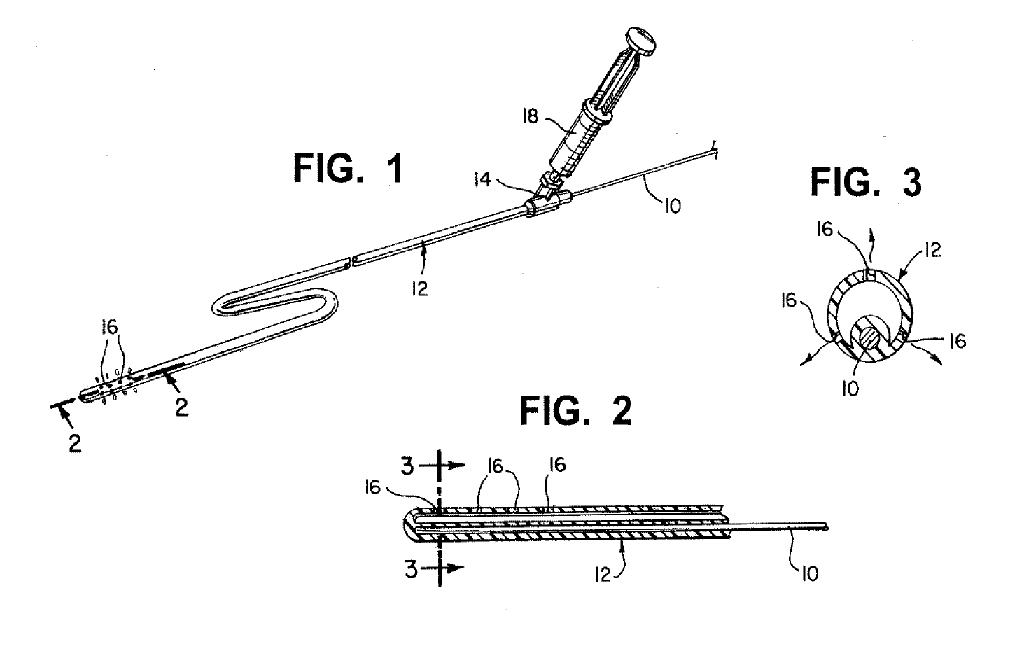

Referring now to FIGS. 1-3, a device and method for removing a hydrophilic coated stylette 10 from an implantable catheter 12 is illustrated. Catheter 12 is, in a currently preferred embodiment, an intraspinal catheter. As shown in FIGS. 1-3, catheter 12 is preloaded with a hydrophilic coated stylette 10. Thus, the hydrophilic coated stylette 10 is placed within catheter 12. A syringe connecter 14, preferably in the form of a luer lock, is connected to the proximal end of catheter 12. A distal end of catheter 12 has at least one opening 16 so that medicament can be delivered to the spine via the catheter. In a currently preferred embodiment, the catheter is pre-loaded with stylette 10, and delivered in this state to the end user.

In a currently preferred embodiment, the end user will connect a syringe 18 to the proximal end of catheter 12 via the luer lock as shown in FIG. 1. Syringe 18 preferably contains pure saline. The saline is injected through the catheter 12 to hydrate the hydrophilic stylette 10. The user can confirm that the stylette is fully wetted by witnessing droplets of saline exiting from distal openings 16, as illustrated in FIG. 1.

The stylette loaded and wetted catheter is then placed at a desired target site within the body. The stylette provides rigidity to the catheter to aid in inserting the catheter, which often has to tunnel through muscle and tissue, at the desired target site. In a currently preferred embodiment, the target site is intraspinal. Once the distal end of catheter 12 is placed at the target site, the hydrophilic coated stylette 10 can be removed from catheter 12. Because the stylette has recently been hydrated, stylette 10 can be removed while maintaining the catheter intact. Thus, the present invention reduces the risk that the location of the distal end of the catheter will be inadvertently displaced, or that the catheter will be accidentally cut during the stylette removal process. Catheter 12 can now be fluidly connected to a medicinal product to deliver the medicinal product to the target site. For example, the proximal end of catheter 12 can be connected to an outlet of an implantable infusion pump so that medicament can be delivered directly to the target site. Of course, one skilled in the art will readily recognize that other devices may be used to deliver medicament to the catheter.

In a currently preferred embodiment, catheter 12 is made of a polymeric material. More preferably, catheter 12 is made of silicone. However, catheter 12 could also be made of polyurethane or other similar biocompatible material as those skilled in the art will readily recognize. Catheter 12 may be reinforced with a structural member that is stiffer than the polymeric material, whether it be silicone, polyurethane or other similar biocompatible material. The reinforce catheter could be made individually or by using a combination of a polymer or metal/alloy. Catheter 12 has an inner surface that may be coated with polytetrafluoroethylene or a hydrophilic material. If the catheter inner surface is coated with a hydrophilic material that hydrophilic material may be the same as the hydrophilic material coated on the stylette. Alternatively, if the catheter inner surface is coated with a hydrophilic material that hydrophilic material may be different from the hydrophilic material coated on the stylette, which when wetted in combination provides added lubricity.

Having described the presently preferred exemplary embodiment of a method of removing a stylette from a catheter in accordance with the present invention, it is believed that other modifications, variations and changes will be suggested to those skilled in the art in view of the teachings set forth herein. Substitutions of elements from one described embodiment to another are also fully intended and contemplated. It is also to be understood that the drawings are not necessarily drawn to scale, but that they are merely conceptual in nature. It is, therefore, to be understood that all such modifications, variations, and changes are believed to fall within the scope of the present invention as defined by the appended claims.

Every issued patent, pending patent application, publication, journal article, book or any other reference cited herein is each incorporated by reference in their entirety.

What is claimed is:

1. A method of removing a hydrophilic coated stylette from an implantable catheter, said method comprising the steps of:
placing the hydrophilic coated stylette within a catheter;
wetting the hydrophilic coated stylette; and
removing the hydrophilic coated stylette from the catheter.

2. The method according to claim 1, wherein the wetting step occurs after the placing the hydrophilic coated stylette within a catheter step.

3. The method according to claim 1, wherein the removing step occurs after the wetting step.

4. The method according to claim 1, wherein the catheter is made of a polymeric material.

5. The method according to claim 4, wherein the catheter is made of silicone.

6. The method according to claim 4, wherein the catheter is made of polyurethane.

7. The method according to claim 1, wherein the catheter is reinforced with a structural member that is stiffer than the polymeric material.

8. The method according to claim 5, wherein the catheter is reinforced with a structural member that is stiffer than silicone.

9. The method according to claim 6, wherein the catheter is reinforced with a structural member that is stiffer than polyurethane.

10. The method according to claim 1, wherein the removing step occurs while maintaining the catheter intact.

11. The method according to claim 1, further comprising the step of placing the stylette loaded catheter at a desired target site within the body.

12. The method according to claim 11, wherein the placing the stylette loaded catheter step occurs after the wetting step.

13. The method according to claim 12, wherein the target site is intraspinal.

14. The method according to claim 12, further comprising, after the removing step, the step of fluidly connecting the catheter to a medicinal product to deliver the medicinal product to the target site.

15. The method according to claim 12, wherein the wetting step occurs after the placing step.

16. The method according to claim 15, wherein the catheter is made of a polymeric material.

17. The method according to claim 16, wherein the catheter is made of silicone.

18. The method according to claim 17, wherein the catheter is reinforced with a structural member that is stiffer than silicone.

19. The method according to claim 15, wherein the removing step occurs while maintaining the catheter intact.

20. The method according to claim 1, wherein the catheter has an inner surface that is coated with polytetrafluoroethylene.

21. The method according to claim 1, wherein the catheter has an inner surface that is coated with a hydrophilic material.

22. The method according to claim 21, wherein the catheter inner surface is coated with a hydrophilic material that is the same as the hydrophilic material coated on the stylette.

23. The method according to claim 22, wherein the catheter inner surface is coated with a hydrophilic material that is different than the hydrophilic material coated on the stylette.
